(19) United States
(12) Patent Application Publication
(10) Pub. No.: US 2005/0096850 A1
(43) Pub. Date: May 5, 2005

(54) Method of processing gene expression data and processing program

(75) Inventor: Tomokazu Konishi, Akita (JP)

Correspondence Address: SEED INTELLECTUAL PROPERTY LAW GROUP PLLC, 701 FIFTH AVE, SUITE 6300, SEATTLE, WA, 98104-7092, US

(73) Assignee: Center for Advanced Science and Technology Incubation, Ltd., Chiyoda-ku (JP)

(21) Appl. No.: 10/702,108

(22) Filed: Nov. 4, 2003

(51) Int. Cl. G06F 19/00; G01N 33/48; G01N 33/50

(52) U.S. Cl. 702/20

(57) ABSTRACT

Gene expression data obtained from a DNA chip or a like chip are analyzed more precisely using a process that includes a sorting/pick-up processing unit that sorts the data values of the obtained array data and picks up a predetermined number of data values from the sorted data values, each at a predetermined interval from each other, a background candidate calculation unit 32 that selects a plurality of background candidates, the values of the background candidates are subtracted from the data values that are picked up, and the subtracted values that are obtained are subjected to a logarithmic conversion, and a difference calculation/comparison processing unit that calculates normal distribution standard values corresponding to the logarithmic values, and calculates indexes of differences between the logarithmic values and the standard values for each of the background candidates. In addition, the range of background candidate values are narrowed based on the indexes, the subtracted values and logarithmic values are obtained, the indexes of the differences are calculated, and the background candidate values are narrowed repeatedly to determine the background value.

### BACKGROUND OF THE INVENTION

1. Field of the Invention

This invention relates to a method of statistically analyzing gene expression data and a program for carrying out the method with a computer.

2. Description of the Related Art

It has been known to utilize a DNA chip for obtaining gene expression data. The DNA chip is the one obtained by fixing a plurality of genes on different spots on a base member such as a slide glass. For example, a microarray includes several thousands to several tens of thousands of genes secured as targets. Targets can be DNAs and mRNAs of a single chain. As the base of the DNA chips, there can be used a glass plate with various coatings, a film of nylon or nitrocellulose, a hollow yarn, a semiconductor material, a metal material or an organic material, which are capable of holding nucleic acids. The target, further, can be the whole cDNA or a partial copy thereof, a partial copy of genome DNA, and synthetic DNA and/or synthetic RNA. In order to secure the target on the base member, there has been known a method of synthesizing oligo DNA on a glass plate by the photolithography method and a method of attaching a target to the base member by utilizing a spotter or the like.

The DNA chip is hybridized with DNA or RNA (to be analyzed) to which has been attached a fluorescent label . The object to be analyzed complementary to the target forms a double chain. Since the fluorescent label has been subjected to the object to be analyzed, image data of the DNA chip can be obtained by using a fluorescent scanner after the hybridization. This makes it possible to learn in which spot the double chain has been formed based on the thus obtained image data. More concretely, the obtained image displays spots stemming from the DNAs as a result of hybridization. Upon integrating the signal intensities of a predetermined region including the spot positions, therefore, it is possible to obtain array data of values of the signal intensities of the spots.

For example, it is possible to obtain the array data showing many gene expressions by just one experimental operation by using a microarray to which several thousands to several tens of thousands of targets have been secured. Therefore, in measuring an increase or a decrease of data of a gene expression, it is a general practice to average the data (values of the signal intensities) of many gene expressions as an object and to standardize the data based thereupon. More concretely speaking, the data are standardized prior to comparing the expression data for each of experiments. An example of standardization has been disclosed in, for example, Johhanes Schuchhardt et al., “Normalization strategies for cDNA microarrays” (Nuclei Acids Research, 2000, Vol. 28, No. 10).

The probability distribution of the obtained data is non-parametric. In order to standardize the obtained data, however, there is employed such a method as Z-standardization, t-standardization, or dividing the integrated value of signal intensities of the spots by an arithmetical mean of all the numerical values as disclosed in, for example, Todd Richmond et al., “Chasing the dream: plant EST microarrays” (Current Opinion in Plant Biology, 2000, Vol. 3, pp. 108-116).

Since they are not the non-parametric methods, the standardization causes significant deterioration of the precision of data.

Further, the array data based on the image obtained by the fluorescent scanner necessarily contain background components. This stems from the fact that the signal intensity of the background existing in the whole image data and the range of measurement do not necessarily comply with the size and the shape of the real spot. For the correct analysis, therefore, it is important to obtain the data of true signal values by subtracting the background component from the value of the obtained image data. The same holds even for the array data obtained by other methods such as detecting the electric signals or detecting radiation.

So far, the background components have been estimated by finding the average value or a median value for each pixel based on numerical values of the signal intensity of a particular spot and that of a portion other than a spot, and by multiplying the obtained value by the number of pixels in the measured region.

There has also been known a method of estimating the background component from the values near the external sides of the measuring range for each of the spots as proposed by Michael Eisen, “ScanAlyze User Manual” (http://rana.lbl.gov/EisenSoftware.htm).

According to the above conventional correction method, however, the estimated value of the background varies depending upon the difference in values in the region of the spot or of the image used for calculating the background value. Namely, various different background values may be calculated, making it difficult to judge which one is correct. In particular, a difference in the background value often increases between a region where the DNA is spotted and a region where it is not spotted.

### SUMMARY OF THE INVENTION

The disclosed embodiments of the present invention are directed to the logarithmic values of data (data of the quantity of light emission due to gene expression) obtained from the DNA chip and three-parameter normally distributed, and subjecting the above data to the logarithmic conversion and to further standardize (e.g., z-standardize) the data. As a result of the above method, the results of different experiments and the results of experiments of the same kind can be accurately compared.

In accordance with one embodiment of the present invention, a data processing method capable of conducting an analysis based on the gene expression data obtained from the DNA chip and the like while maintaining good precision is provided.

A method of processing gene expression data to obtain data that can be analyzed by processing array data obtained based on the amount of expression of genes, such as processing the array data constituted by values of signal intensities of spots arranged on the chip by the hybridization of a DNA chip or a protein chip is provided. The method includes the steps of:

obtaining the array data, sorting the data values of the obtained array data, picking up a predetermined number of data values from locations at predetermined intervals, and temporarily storing them in storage means;

selecting a plurality of background candidates and temporarily storing them in the storage means;

subtracting the values of the background candidates from the data values that are picked up to obtain subtracted values, obtaining logarithmic values by subjecting the subtracted values to logarithmic conversion, and temporarily storing the logarithmic values in the storage means;

calculating standard values of the normal distribution corresponding to the logarithmic values;

calculating indexes of differences between the logarithmic values and the standard values for the background candidates;

narrowing the range of the background candidate values based on the indexes;

obtaining the subtracted values and logarithmic values, calculating the indexes of the differences, and narrowing down the background candidate values repetitively, and finally determining the background value; and

standardizing the logarithmic values temporarily stored by relating them to the determined background value, and storing the standardized values in the storage means.

According to one aspect of the present invention, a background value is determined based on a difference between a logarithmic value of the sorted value and a corresponding standard value to render the difference to be minimal. It is thereby possible to determine a more suitable background value and, hence, to obtain more proper data for analysis including comparison with other data.

As an index of difference, there can be used the sum of absolute values of differences, the sum of squares of differences (square errors), and “r” in the method of least squares. In picking up a predetermined number of data values from the sorted data values of locations at predetermined intervals, the predetermined interval may be the interval “0”, i.e., all data may be picked up. Among the data of a number of n that are picked up, the standard value corresponding to the i-th data value may be the value of i-th part of the normal distribution which has been divided into n parts.

Further, another aspect of the invention is provided by a method of processing gene expression data to obtain data that can be analyzed by processing array data obtained based on the amount of expression of genes, the method including the steps of:

obtaining the array data, sorting the data values of the obtained array data, picking up a predetermined number of data values from the sorted data values of locations at predetermined intervals, and temporarily storing them in storage means;

determining a background value ν and storing it in the storage means;

converting subtracted values, which are the data values from which the background value is subtracted, into a logarithmic form to obtain logarithmic values, and temporarily storing them in the storage means;

referring to the logarithmic values to calculate a characteristic value μ describing the central tendency and a characteristic value a describing the variation, and storing them in the storage means; and

calculating z=(log (x−ν)−μ)/σ as standard values z for the data values x, and storing the calculated standard values z in the storage means.

According to the present invention, the data values x of array data are standardized as z=(log (x−ν)−μ)/σ by using the calculated parameters ν, μ and a so as to obtain the values more suited for the analysis.

According to a preferred embodiment, the step of determining the background value ν includes the steps of:

selecting a plurality of background candidates and temporarily storing them in the storage means;

subtracting the values of the background candidates from the data values that are picked up to obtain subtracted values, obtaining logarithmic values by subjecting the subtracted values to logarithmic conversion, and temporarily storing the logarithmic values in the storage means;

calculating normal distribution standard values corresponding to the logarithmic values;

calculating indexes of differences between the logarithmic values and the standard values for each of the background candidates;

narrowing the range of the background candidate values based on the indexes; and

repeatedly obtaining the subtracted values and logarithmic values, calculating the indexes of the differences, and narrowing the background candidate values, and thereby determine the background value.

According to a more preferred embodiment, the step of calculating a characteristic value μ of central tendency and a characteristic value σ of variation includes the steps of:

calculating standard values corresponding to the logarithmic values;

comparing the logarithmic values with the standard values to find a range in which the ratio of the two shifts nearly at a constant rate;

calculating the slope of a straight line formed in the above range, where the standard value is the x-axis and the logarithmic value is the y-axis, as well as calculating the y-intersect; and

making the calculated y-intersect the characteristic value μ of central tendency and making the slope the characteristic value σ of variation.

Here, the so-called normal probability plot (NPP) is utilized to find a region where the linearity is maintained and to determine the slope of the straight line derived from the region and the intersect to be σ and μ, respectively. This makes it possible to realize a more robust standardization.

Another preferred embodiment includes the steps of:

rearranging the order of data values from the order of spots arranged on the chip, and temporarily storing them in this order in the storage means;

calculating the indexes of the variation pattern in data values within each of the columns or rows of spots arranged in the chip;

calculating the median value of data values for each of the columns or rows based on the indexes when there is a change tendency in that column or row; and

dividing the data values by the corresponding median values to obtain divided values, and temporarily storing them in the storage means;

wherein the divided values which are temporarily stored are used for the operation as values corresponding to the data values of the array data.

This embodiment eliminates the singularity even when there is a problem in the precision of the array chip, particularly, when the columns and rows have distinct characteristics due to problems in the precision of the engraving machine, or due to the generation of clones arranged at the spots of the chip itself, and establishes a state making robust standardization possible.

The step of calculating the index that represents the change tendency may further include a step of calculating the average change of values in a particular column or row.

Another preferred embodiment utilizes the steps of:

Further rearranging the order of data values from the order of spots arranged on the chip, and temporarily storing them in this order in the storage means;

finding a periodicity of data values in the above order; and

calculating subtracted values by subtracting the characteristic value of central tendency of the period from all the data values where there is periodicity, and temporarily storing them in the storage means;

wherein the temporarily stored subtracted values are used for the operation as values corresponding to the data values of the array data.

Here, when the values of array data have a predetermined periodicity, and the elements having periodicity are excluded to obtain the data that are more suited for being analyzed.

Another preferred embodiment has the steps of:

rearranging the order of data values from the order of spots arranged on the chip;

calculating characteristic values of central tendency of data values of each of the columns or rows of spots arranged in the chip;

setting background values of the spots of each column and row based on the characteristic value of central tendency, and calculating subtracted values by subtracting the background values from the data values of the spots;

converting the subtracted values into a logarithmic form to obtain logarithmic values; and

subtracting characteristic values of central tendency of said logarithmic values of the columns or rows and temporarily storing the subtracted values in the storage means;

wherein the temporarily stored subtracted values are used for the operation as values corresponding to the values of the array data.

In accordance with a further aspect of the present invention, a method of processing gene expression data to obtain data that can be analyzed by processing array data obtained based on the amount of expression of genes is provided, including the steps of:

calculating the characteristic value of central tendency of data values of each of the columns or rows where the spots are arranged in the chip;

setting a candidate for the background value of the spots of the column or the row based on the characteristic value of central tendency, and calculating a subtracted value by subtracting the background candidate value from the data values of the spot;

converting the subtracted values into a logarithmic form to obtain logarithmic values;

calculating a characteristic value of central tendency of the logarithmic values of the column or the row, and subtracting the characteristic value from the logarithmic values to calculate the second subtracted values;

dividing the data values of each column or row by the characteristic value of variation calculated based on the second subtracted value of the column or the row to obtain divided values, and temporarily storing them in the storage means;

comparing the divided values with the corresponding standard values, and making the background candidate value which minimizes the index of difference between them the background value ν; and

storing the background value ν, and then the characteristic value μ of central tendency and the characteristic value a of variation corresponding to the background value vin the storage means.

According to the present invention, the background value is determined based on the characteristic value of central tendency for each of the columns or rows. For example, the background value for each of the columns can be considered to be proportional to the characteristic value of the central tendency of the column. This makes it possible to eliminate the distinctive difference of the columns or rows.

In accordance with yet a further embodiment of the present invention, a method of processing gene expression data to obtain data that can be analyzed by processing array data obtained based on the amount of expression of genes is disclosed to include the steps of:

obtaining the array data, sorting the data values of the obtained array data, and temporarily storing the sorted data in the storage means;

calculating normal distribution standard values corresponding to the sorted data values;

setting a characteristic value s of variation of the data values, storing it in the storage means, and multiplying the standard values by the characteristic value s of variation to obtain multiplied values;

comparing the data values with the multiplied values to find a range in which the ratio of the two changes at a constant rate;

calculating the slope of a straight line formed in the above range, where the multiplied value is the x-axis and the logarithmic value is the y-axis and calculating the y-intersect; and

making the natural logarithm of the slope the characteristic value μ of central tendency and making the y-intersect the background value g, and storing them in the storage means.

For example, when the noise level of the hybridization as a whole is heightened due to defect in the wet test and cannot be neglected, the standardization can be accomplished based on a combination of the chip and the sample data. When there is no noise and a normal logarithmic distribution can be expected, the standardization can be accomplished by utilizing the above method.

Here, it is desired that the method further include the steps of:

solving xi in compliance with,
xi=(10^{u}) (10^{(sZi)})+g

where Zi is an i-th standard value,

and temporarily storing it in the storage means; and

finding a lower limit value that can be used as xi and storing it in the storage means.

This makes it possible to learn the range of data that can be utilized as an object of analysis.

Further, a program that can be read by a computer to operate the computer to obtain data that can be analyzed by processing the array data obtained based on the amount of expression of genes is provided. The program has the computer execute the steps of:

obtaining the array data, sorting the data values of the obtained array data, picking up a predetermined number of data values from the sorted data values each at a predetermined interval from each other, and temporarily storing them in storage means;

selecting a plurality of background candidates and temporarily storing them in the storage means;

subtracting the values of the background candidates from the data values that are picked up to obtain subtracted values, obtaining logarithmic values by subjecting the subtracted values to the logarithmic conversion, and temporarily storing the logarithmic values in the storage means;

calculating normal distribution standard values corresponding to the logarithmic values;

calculating indexes of differences between the logarithmic values and the standard values for the background candidates;

narrowing the range of the background candidate values based on the indexes;

obtaining the subtracted values and logarithmic values, calculating the indexes of the differences between these, and narrowing the background candidate values, repetitively, to determine the background value; and

standardizing the logarithmic values temporarily stored by relating them to the determined background value, and storing the standardized values in the storage means.

Also disclosed is a program that can be read by a computer to operate the computer to obtain data that can be analyzed by processing the array data obtained based on the amount of expression of genes. The program includes having the computer execute the steps of:

obtaining the array data, sorting the data values of the obtained array data, picking up a predetermined number of data values from the sorted data values each at a predetermined interval from each other, and temporarily storing them in storage means;

determining a background value ν and storing it in the storage means;

converting subtracted values which are the data values from which the background value is subtracted into a logarithmic form to obtain logarithmic values, and temporarily storing them in the storage means;

referring to the logarithmic values, calculating a characteristic value μ of central tendency and a characteristic value σ of variation, and storing them in the storage means; and

calculating z=(log (x−ν)−μ)/σ as standard values z for the data values x, and storing the calculated standard values z in the storage means.

The present invention also provides a program that can be read by a computer to operate the computer to obtain data that can be analyzed by processing the array data obtained based on the amount of expression of genes, the program working to have the computer execute the steps of:

calculating characteristic values of central tendency of data values of the columns or rows on where spots are arranged in the chip for each of the columns or rows;

setting candidates of background values of the spots belonging to the column or the row based on the characteristic values of central tendency, and calculating subtracted values by subtracting the background candidate values from the data values of the spots;

converting the subtracted values into a logarithmic form to obtain logarithmic values;

calculating characteristic values of central tendency of the logarithmic values of the columns or the rows and subtracting the characteristic values from the logarithmic values to calculate second subtracted values;

obtaining divided values by dividing the data values by the characteristic value of variation calculated based on the second subtracted values of the column or the row, and temporarily storing them in the storage means;

comparing the divided values with the corresponding standard values and making the minimum index of difference between them the background value νν; and

storing the background value ν, and the characteristic value μ of central tendency and the characteristic value σ of variation of the background value ν in the storage means.

In accordance with another embodiment of the present invention, a program is disclosed that can be read by a computer to operate the computer to obtain data that can be analyzed by processing the array data obtained based on the amount of expression of genes, the program working to have the computer execute the steps of:

obtaining the array data, sorting the data values of the obtained array data, and temporarily storing the sorted data in the storage means;

calculating normal distribution standard values corresponding to the sorted data values;

setting a characteristic value s of variation of the data values, storing it in the storage means, and multiplying the standard values by the characteristic value s of variation to obtain multiplied values;

comparing the data values with the multiplied values to find a range in which the ratio of the two shifts at a constant rate;

calculating the slope of the straight line formed in the above range when the multiplied value is considered to be the x-axis and the logarithmic value is the y-axis and calculating a y-intersect; and

making the natural logarithm of the slope the characteristic value μ of central tendency and the intersect as a background value g, and storing them in the storage means.

As the base member of a DNA chip, there can be used a plate made of a glass on which various coatings are applied, a film made of such a base member as nylon or nitrocellulose, a hollow yarn, a semiconductor, a metal, or an organic material, that is capable of holding nucleic acid on the surfaces thereof. On the DNA chip, further, there is arranged, as a target, the whole cDNA or a copy of a portion thereof, a copy of genome DNA, a synthetic DNA or a synthetic RNA.

To prepare the chip, further, a nucleic acid is prepared and is adsorbed or is bonded by static electricity, or is arranged on the base member by covalent bond, or the nucleic acid is synthesized on the base member. Signals of the signal intensity can be detected by an electric method by utilizing a semiconductor chip or by a method of detecting fluorescence or radioactivity.

The disclosed embodiments of the present invention can be applied to the array data from the DNA chip having any of the above targets formed on any of the above base members. The invention can be further applied to the array data obtained by using any method. The same also holds for the data obtained from other media, such as microbeads, to which are fixed genes such as fixed DNA.

In the present invention, the DNA chip includes any one such in which a nucleic acid is arranged on the base member, such as RNA chip forming RNA on the base member, microarray, macroarray, dot-blot, reversed northern, etc.

### BRIEF DESCRIPTION OF THE DRAWINGS

FIG. 1 is a diagram of a hardware constitution of an analyzer according to a first embodiment of the invention;

FIG. 2 is a functional block diagram of major portions of the analyzer according to the first embodiment;

FIG. 3 is a flowchart schematically illustrating the processing by the analyzer according to the first embodiment;

FIG. 4 is a flowchart illustrating, in detail, the processing for calculating a background value according to the first embodiment.

FIG. 5 is a flowchart illustrating a processing for calculating a parameter according to the first embodiment;

FIG. 6 is a flowchart illustrating an initial correction processing according to the first embodiment;

FIG. 7 is a flowchart illustrating the initial correction processing according to the first embodiment;

FIG. 8 is a flowchart schematically illustrating the processing according to a second embodiment;

FIG. 9 is a flowchart schematically illustrating the processing according to the second embodiment;

FIG. 10 is a flowchart schematically illustrating the processing executed by the analyzer according to a third embodiment;

FIG. 11 is a flowchart illustrating another example of the initial correction processing according to a further embodiment of the invention;

FIG. 12 is a graph illustrating an example of indexes of differences for the background candidates;

FIG. 13 is a graph illustrating an example of indexes of differences for the background candidates;

FIG. 14 is a graph plotting the values with the ideal values (theoretical values) as the abscissa and data values that are found as the ordinate;

FIG. 15 is another graph plotting the values with the ideal values (theoretical values) as the abscissa and data values that are found as the ordinate;

FIG. 16 is a graph illustrating data values for each of the data spots obtained from a given DNA chip and average values of variation; and

FIG. 17 is a graph plotting the values with (10^{(sZi)}) as the x-axis and xᵢ as the y-axis of the data stemming from a given DNA.

### DETAILED DESCRIPTION OF THE INVENTION

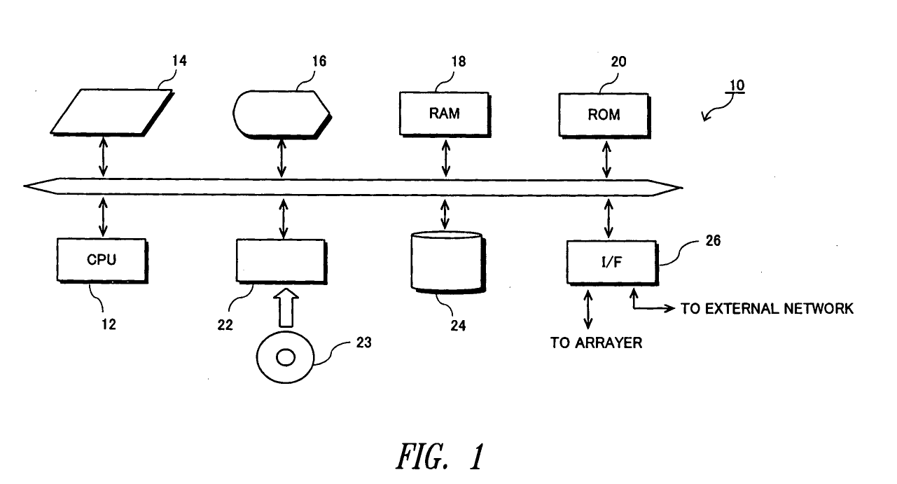

Embodiments of the invention will now be described with reference to the accompanying drawings. FIG. 1 is a diagram illustrating the hardware constitution of an analyzer according to a first embodiment of the present invention. Referring to FIG. 1, the analyzer 10 includes a CPU 12, an input unit 14 such as a mouse or a keyboard, a display unit 16 constituted by a CRT, a RAM (random access memory) 18, a ROM (read only memory) 20, a portable storage medium driver 22 accessible by a portable storage medium 23 such as CD-ROM or DVD-ROM, a hard disk unit 24, and an interface (I/F) 26 for controlling the exchange of data relative to the external unit. As will be comprehended from FIG. 1, a personal computer or the like can be used as the analyzer 10 of the embodiment.

The I/F 26 is connected to a reader or a scanner (not shown) that measures the amount of light emitted by a spot on the hybridized DNA chip and forms the data based on the measured amount of emitted light, and is further connected to a communication circuit. The communication circuit is further connected to an external network (e.g., Internet).

In this embodiment, the portable storage medium 23 is storing a program that receives data from the reader or the scanner and executes a necessary data conversion processing that will be described later for the data, as well as a program for analyzing the data that are processed. Therefore, the portable storage medium driver 22 reads the program from the portable storage medium 23, stores it in the hard disk unit 24 to start it, whereby the personal computer works as the analyzer 10. Or, the program may be down-loaded via an external network, such as an internet.

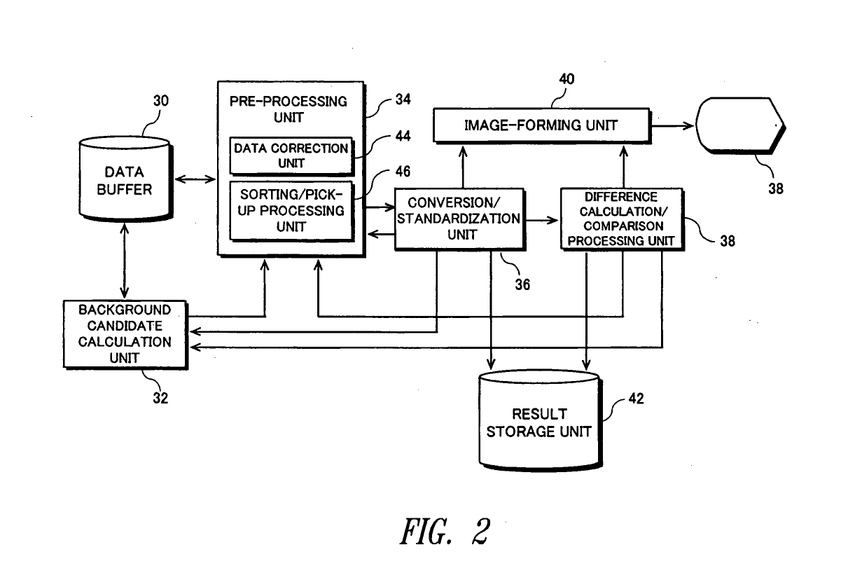

FIG. 2 is a functional block diagram of major portions of the analyzer 10 according to the first embodiment. FIG. 2 illustrates a constituent portion for executing a process for deriving analytical results of the gene expression data. Referring to FIG. 2, the analyzer 10 includes a data buffer 30, a background candidate calculation unit 32 for calculating the candidates of background values corresponding to noise components in the amount of light emitted by a spot on the DNA chip based on the data (original data) temporarily stored in the data buffer 30, a pre-processing unit 34 that executes a predetermined pre-processing for the original data and executes the operation between the background candidate values and the original data, a conversion/standardization processing unit 36 for standardizing the converted data, a difference calculation/comparison processing unit 38 that calculates a difference between a standardized value and an ideal value, compares the differences of a plurality of background candidates, and calculates corrected values of a graph based on the compared results, an image-forming unit 40 for forming an image to be offered to the user, and a result storage unit 42 for storing a variety of data that are obtained.

The pre-processing unit 34 includes a data correction unit 44 that executes a process for enhancing the randomness in case the original data have regularity in the column and position (region) of the DNA chip, and a sort/pick-up processing unit 46 for sorting the data corrected, as required, by the data correcting unit 44, and for picking up predetermined data from the group of sorted data.

The function of the data buffer 30 is realized by the RAM 18 or, depending upon the cases, by the hard disk unit 24. The data buffer temporarily stores the data of the amounts of light emitted by the spots transmitted from the reader or the scanner, or temporarily stores the data of the amounts of light emitted by the spots that had been transmitted from the reader or the scanner and had been stored in advance in a predetermined region of the hard disk unit 24. Further, the data buffer 30 is capable of temporarily storing the background candidate values calculated by the background candidate calculation unit 32, the data processed by the pre-processing unit 34 and, depending upon the cases, the data subjected to the logarithmic conversion as well as standard values or ideal values used for the operation.

The reader or the scanner images the DNA chip through a CCD camera, integrates the signal intensities for each of the spots, and produces them as array data. Alternatively, the reader or the scanner determines a background value based upon the data value of the image taken by the CCD camera, subtracts the background value from the signal intensities of the pixels, integrates the signal intensities for each of the spots based on the image data which has been corrected for background, and outputs them as array data. This embodiment is capable of utilizing unprocessed array data or the data that are corrected (corrected for the background) by the reader, the scanner, or the accompanying software. In this specification, the data obtained by accumulating the signals for each of the spots transmitted from the reader or the scanner are referred to as “original data” in the sense that they serve as array data or as data for effecting the background processing according to the embodiment.

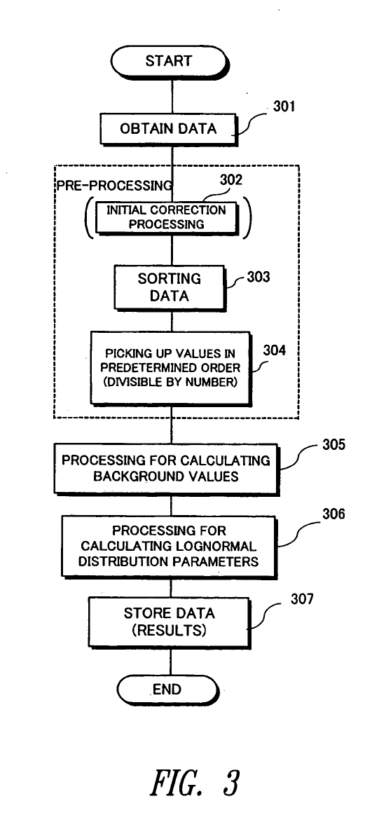

Described below in detail is a process performed by the analyzer 10 for calculating an index that can be compared with other data based on the data of the amount of light emission expressed on the DNA chip. FIG. 3 is a flowchart schematically illustrating the processing by the analyzer 10 according to this embodiment. Referring to FIG. 3, the analyzer 10, first, obtains the original data of the DNA chip from the data buffer 30 (step 301) and executes the pre-processing therefor (see step 310). In this embodiment, the pre-processing includes an optional initial correction processing (step 302) executed, as required, based on the state of the original data, sort processing (step 303) for sorting the obtained original data, and pick-up (step 304) of data values positioned in predetermined order in the sorted data group. The initial correction processing will be described later in detail.

For the data rearranged in order of increasing values or decreasing values through the sort processing, the sort/pick-up processing unit 46 in the processing unit 34 picks up the data positioned in order, maintaining a predetermined interval. For example, the values at predetermined positions may be picked up maintaining such an interval as 10-th, 20-th, 30-th, - - - in decreasing order. Alternatively, predetermined positions may be picked up, such as a position divisible by 100, a position divisible by 200, - - - . The sorted data and the picked-up data are stored in a predetermined region of the data buffer 30.

Then, a background value is calculated (step 305) and other parameters are calculated (step 306). In this embodiment, a group of data that are more robustly standardized are produced from the data of a given DNA chip by taking lognormal distributions of values of data (data of the amounts of light emitted by the gene expression) obtained from the DNA chip and applying z-standardization, making it possible to correctly compare the results of different experiments or the results of the same kind of experiments.

In this embodiment, the calculated background value is denoted by ν and is chosen from among values of z=(log (x−y)−μ)/σ, and the remaining parameters μ and σ are calculated by the operations that will be described later. First, calculation of the background values will be described in further detail and, then, calculation of the remaining parameters will be described in detail.

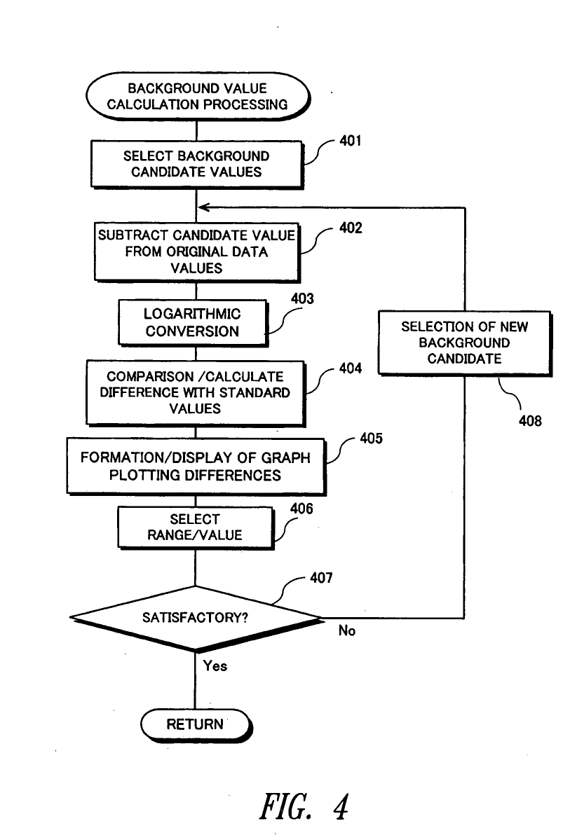

FIG. 4 is a flowchart illustrating the background value calculation processing (step 305) in further detail. The background candidate calculation unit 32 determines a range of background value candidates (background candidate values) and a plurality of background candidate values in that range depending upon the input by the operator who manipulates the input unit. For example, the user may specify a start point (e.g., “0 (zero)”) of a background candidate value and an end point (e.g., median value or first quarterly position). Then, values whose number is predetermined are determined maintaining an equal interval (or maintaining a geometrical ratio) between the start point and the end point. When “0” and a median value are specified, for example, eight values are obtained maintaining an equal distance, and ten background candidate values are determined inclusive of the start point and the end point. In this processing, the background candidate values are stored in the data buffer 30 and, as required, the values are read out or updated.

Then, a given background candidate value is subtracted from the value of original data (original data value) that is picked up (step 402), and the original data value from which the background candidate value has been subtracted is subjected to the logarithmic conversion by the conversion/standardization processing unit 36 (step 403). The thus obtained data, which has been subjected to the logarithmic conversion, and are also stored in the data buffer 30 for being used in the subsequent process steps 402 and 403, are executed for all of the selected (e.g., ten) background candidate values.

Next, a given background candidate value subjected to the logarithmic conversion (converted value) is compared with a corresponding standard value calculated by a method described below and stored in the data buffer 30, to calculate an index of a difference in the value (step 404). Here, in this embodiment, the standard value is calculated as described below.

Since there is an interval between measured values, the following numerical value is calculated for correcting a statistical median value.
m(i)=(i−0.3175)/(n+0.365)
where n is a number of data items, and i is a natural number from 1 to n.

Next, an inverse function F⁻¹(r) of the normal distribution function is applied to the m(i) that is calculated. The values that are calculated become standard values corresponding to the data values.

Next, the difference calculation/comparison processing unit 38 calculates the sum of absolute values of, for example, differences (differences between the data values and the standard values) of the background candidate values, or calculates the sum of squares of differences. The values calculated here serve as indexes of differences of the background candidate values. It is allowable to use “r” of the method of least squares as an index of difference, as a matter of course. In practice, it is desirable to utilize “r” of the method of least squares from the standpoint of finding highly precise background values.

Next, the difference calculation/comparison processing unit 38 describes a graph with the background candidate value as the abscissa and the index of difference as the ordinate, and displays it on the screen of the display unit 16 (step 405).

The operator makes a reference to the graph displayed on the screen of the display unit 16, and selects a desired range of background candidate values or selects background values (step 406). When the selected values are considered to be satisfactory as background values (yes at step 407), the processing ends. When the selected values are not satisfactory, a predetermined number of new background candidate values are determined from a narrowed range of background candidate values (step 408), and the processing of steps 402 to 407 is repeated. The new background candidate values may be those obtained by equally dividing the distance between the start point and the end point of the range of background candidate values, or may be those obtained by dividing the distance by a geometrical ratio. The finally obtained background values are stored in the result storage unit 42.

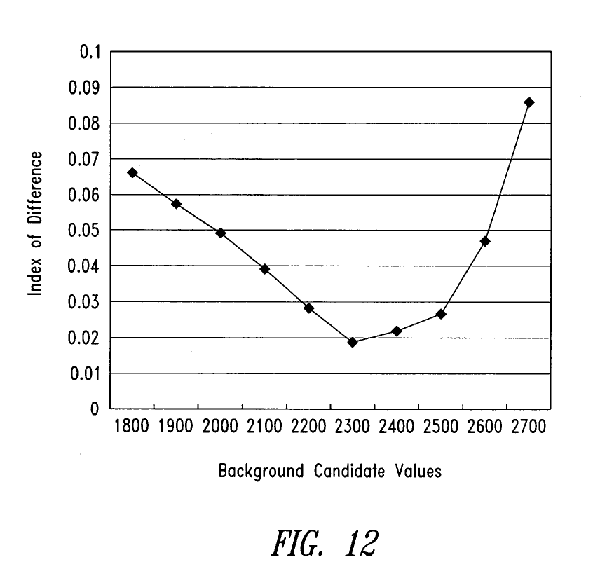

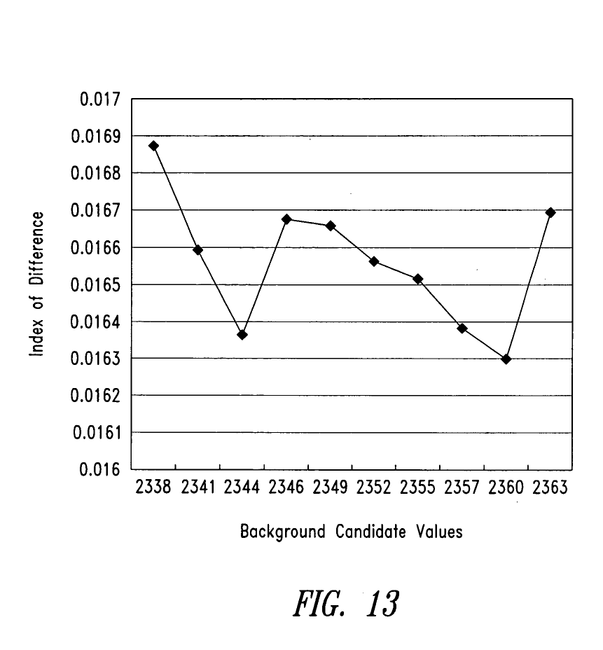

As shown in, for example, FIG. 12, a graph is drawn with the range of background candidate values as the abscissa and the index of difference as the ordinate. In the example of FIG. 12, the background candidate values are (1800, 1900, 2000, - - - , 2700) from 1800 to 2700 maintaining an interval of 100. By referring to these, the observer narrows the range of background candidates, and obtains again the indexes of differences of the background candidate values within the new range (see FIG. 13). In the example of FIG. 13, it can be comprehended that the background value at this moment is most suitably “2363”.

Next, described below is a process for calculating the remaining parameters. In general, in a normal logarithmic distribution s the average value is taken as μ (characteristic value of central tendency) and the standard deviation is taken as σ (characteristic value of variation). In the data obtained from the DNA chip, however, a strong signal (having a relatively large data value) contains correct data and a weak signal (having a relatively small data value) contains a relatively large noise. The logarithmic values of data assuming negative values being concealed by noise cannot be calculated. Therefore, many of these weak signals are discarded away. In this case, it is not allowed to utilize the above-mentioned calculation method.

Usually, an average value is made the characteristic value of central tendency. However, the averaging is not a so-called robust method, and is calculated to be slightly higher particularly under a condition where weak signals are selectively removed. In such a case, it has been known that the median value is more effective.

On the other hand, standard deviation is made the characteristic value of variation . However, the standard deviation is not a robust method, either, and is calculated to be slightly smaller under a condition where weak signals are selectively removed. As a robust method, on the other hand, there has been known iqr for finding a characteristic value of variation from a quartile range (e.g., see http://infoshako.sk.tsukuba.ac.jp/InfoRes/jdoc/MALAB5/jhelp/toolbox/stats/iqr.html).

However, the median value is calculated from one point in the group of data, and iqr is calculated from two points in the group of data, and involve difficulty in regard to the precision. In particular, when the data are obtained from a small number of spots or when there is a limit on the number of data for correction, the problem becomes serious. In this embodiment, therefore, the parameters are calculated by the following method, maintaining a high precision even when there is a limit in the number of data.

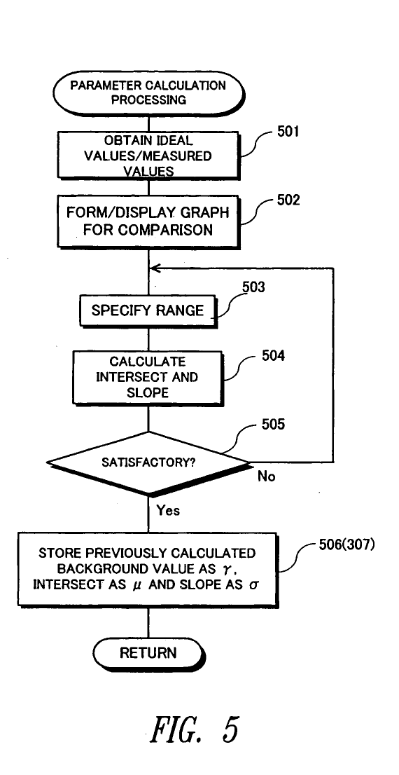

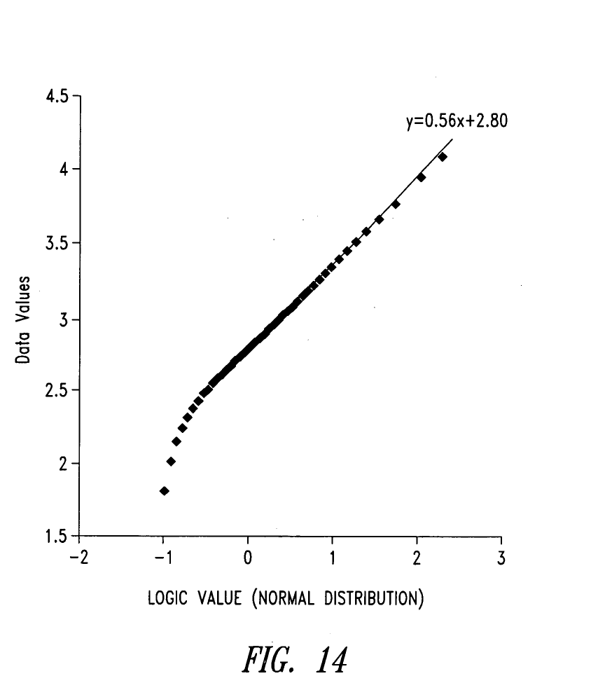

FIG. 5 is a flowchart illustrating the process for calculating parameters according to this embodiment. Referring to FIG. 5, there are obtained an ideal value and a measured value from which the background value is subtracted (step 501). The ideal value is similar to the standard value calculated at step 404. Then, a graph is drawn with the ideal value (theoretical value) on the abscissa and the data value based on the measured value on the ordinate, and is displayed on the screen of the display unit (step 502). In this graph, if the measured values are along the log normal distribution, the graph is nearly in agreement with y=x. In practice, however, as shown in FIG. 14, the graph obtained by plotting the measured values has a slope other than 1 (=a, a≅0.56 in FIG. 14) and a y-intersect (=b, b≅2.80 in FIG. 14), and even loses linearity in a portion where the value of x is relatively small.

Even in the graph of FIG. 14, however, there exists a portion that is recognized to be nearly straight (e.g., portion where x becomes positive). In this embodiment, therefore, if the user operates the input unit making reference to the graph and specifies a range which is judged to possess linearity (step 503), then, a linear equation of the difference between the measured value and the theoretical value is calculated, for example, relying on the method of least squares by using measured values in the specified range. The slope “a” of the primary equation “ax+b” that is calculated corresponds to the characteristic value “σ” of variation, and the y-intersect “b” corresponds to the characteristic value “μ” of central tendency (step 504).

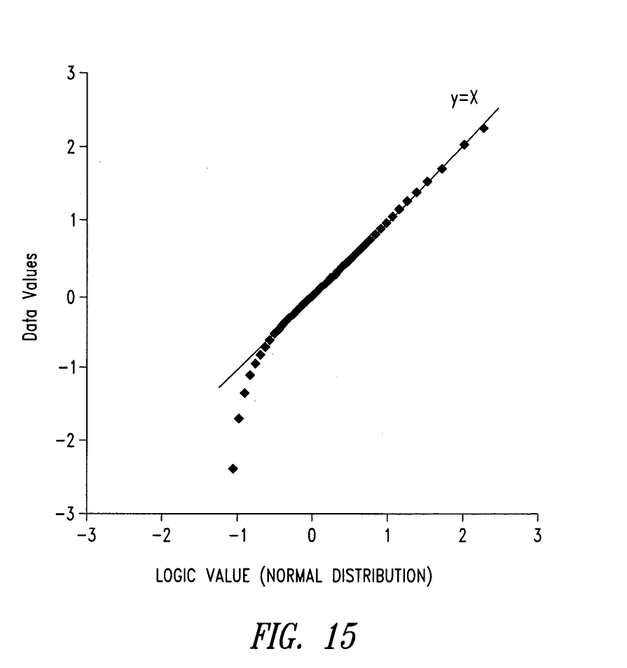

For example, the image-forming unit 40 of the analyzer 10 may form a graph by using “a” and “b” that are calculated and with the theoretical value on the abscissa and the measured value z=(log (x−ν)−μ)/σ on the ordinate, and may display it on the screen of the display unit 38. FIG. 15 is a graph drawn again by plotting the values plotted in FIG. 14 after μ is subtracted therefrom followed by the division by σ. The user makes reference to the graph that is displayed. If it is not satisfactory (no at step 505), the user specifies again the range in the initial graph and executes again the processes of step 503 and of subsequent steps.

If satisfactory (yes at step 505), the background value just calculated is denoted by “ν”, the intersect is denoted by “μ” and the slope by “σ”, and are of the data for specifying the DNA chip and are stored in the result storage unit 42. By using the thus obtained parameters, the data values x obtained from the DNA chip are standardized in compliance with the equation,
z=(log (x−ν)−μ)/σ

According to this embodiment, as described above, a suitable background value is calculated, the effect of noise is precluded, and the characteristic value of central tendency and the characteristic value of variation for the standardization are calculated from the linear portion of the graph drawn by plotting the measured values. It is thus possible to realize a more robust standardization.

Next, the initial correction processing (step 302) according to this embodiment will be described in further detail. In this embodiment, two kinds of correction can be effected depending upon the characteristics of data from the DNA chip.

The DNA chip is formed by a method of engraving the DNA on the surface of a glass or the like. Here, due to the lack of precision of the engraving device (arrayer or spotter), the data values often become “slightly larger” or “slightly smaller” as a repeated tendency.

Such a tendency often is evident in each of the pins of the arrayer, for each of the transverse strings of the spotted grids, or for each of rows and columns of the grid of the microtitre plate for holding the DNA sample.

When the data become strong or weak according to, for example, the transverse row in the grid, a process can be contrived to standardize the data for each transverse row. In this case, however, a set of data is constituted by a decreased number n of data items (e.g., 32 items). When the background value is estimated from such a small number of data and when there are calculated a characteristic value of central tendency and a characteristic value of variation, then, the precision drops to a conspicuous degree. It has been known that a standard deviation possessed by an average value of random numbers vary in inverse proportion to the square root of n. This indicates that it is difficult to correctly foresee the characteristic value of central tendency from a small number of data.

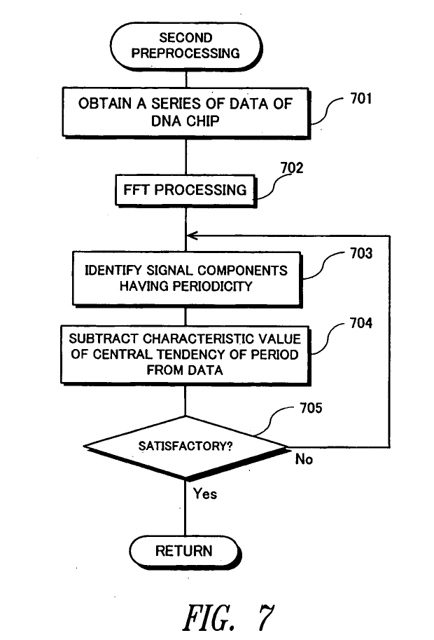

In the initial correction processing, therefore, the value can be corrected for each of the columns (first pre-processes reference numeral 600) if average variation of transverse columns and longitudinal rows of the DNA chip are calculated, and if each of the columns and rows possess characteristics. Even in other cases, the data correction is executed by taking the periodicity into consideration if changes in the values have a periodicity for each of the spots (second correction processes FIG. 7).

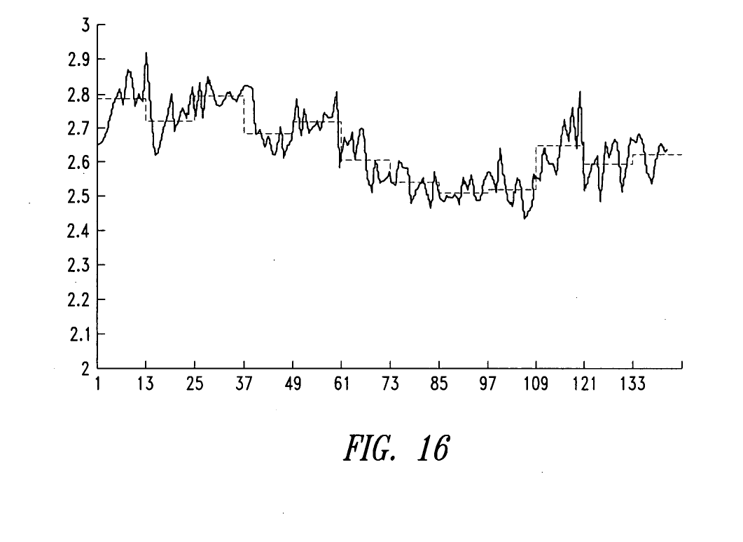

The transverse rows will now be described. Here, needless to say, the similar processing can also be executed for the vertical columns. First, in preparing a DNA chip by using a spotter, the data are arranged in order of really spotting them. Among the-group of such data, there is calculated an average value of data of the rows on the DNA chip and of the rows of a predetermined number preceding and succeeding the above rows (e.g., two preceding and succeeding rows)(steps 601, 602). Calculation of the average value is repeated up to the end of the column (see steps 603, 604) and, then, it is judged whether there is a distinguishing characteristic in the average value for each of the columns (step 605). FIG. 16 is a graph illustrating logarithmic values for each of the spots and average values of variation of the logarithmic values of data obtained from a given DNA chip. In the example shown in FIG. 16, the DNA chip has 32 spots in a transverse string. If the original data values are random as a result of averaging the data values of a predetermined number of preceding and succeeding rows, then, the above average values are nearly in agreement. In the graph of logarithmic values for each of the spots represented by solid lines in FIG. 16, it is not possible to see any variation pattern in the values. However, average logarithmic values of data corresponding to 32 spots of a row are greatly dispersing as indicated by the broken lines. In such a case, it is so judged that a distinctive characteristic resides in each of the columns of the DNA chip (yes at step 605), and the first pre-treatment is executed for the data values.

At step 605, it may be examined whether the dispersion in the average value of variation is significant.

In the first pre-treatment, there is calculated the median value of data values corresponding to the spots of the rows of the DNA chip (step 607), and the data values corresponding to the spots of the columns are divided by the median value (step 607). This is executed for each of the rows (see steps 609, 610).

Next, the second pretreatment will be described. Here, the correction is effected by taking into consideration whether the data values corresponding to the spots are oscillating. First, there are obtained the data values arranged in order of spots (step 701), and an FFT (fast Fourier transform) processing is executed for the group of data (step 702). As a result of FFT, if there are components (signal components) having periodicity, a value of component corresponding to the phase is subtracted from the data values, thus taking the period into consideration (steps 703, 704). The operator may repeat the processes of steps 703 and 704 until satisfactory results are obtained. The data subjected to the first correction treatment or to the second correction treatment are stored in the data buffer 30. The data are subjected to the data sorting (see step 303 in FIG. 3) and to the subsequent processes.

According to the initial correction processing of the embodiment as described above, it is made possible to preclude regularity at the time of preparing the spots.

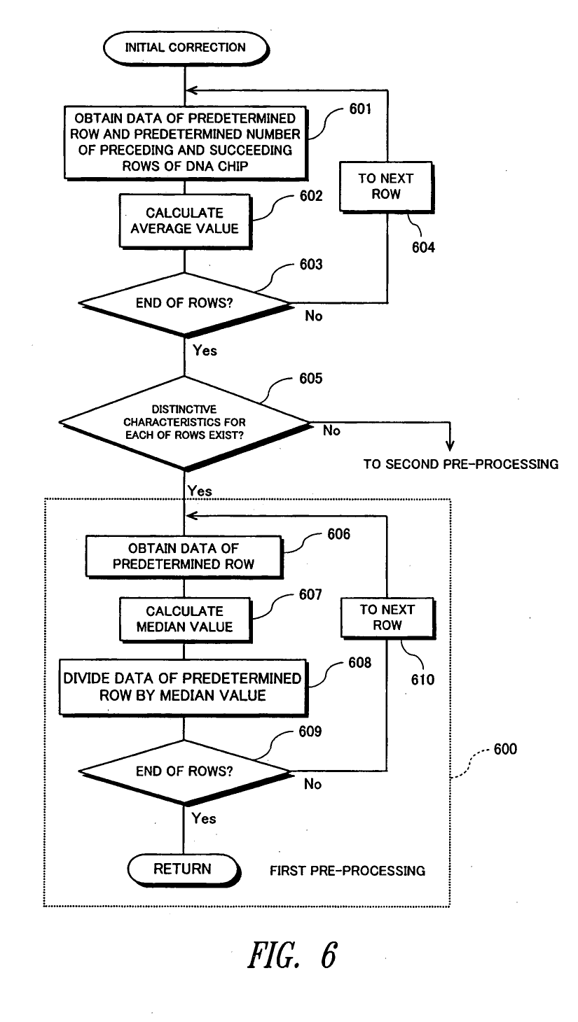

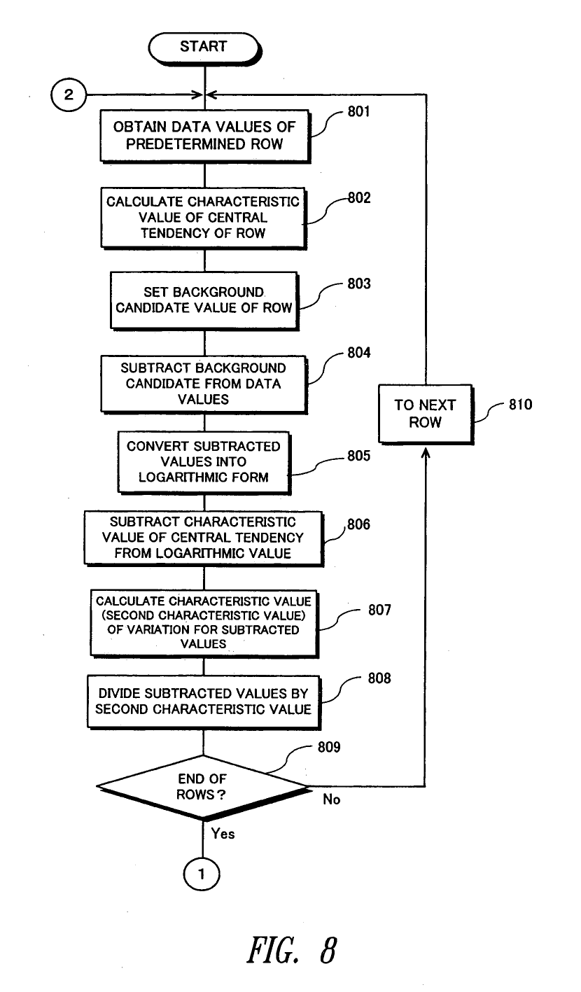

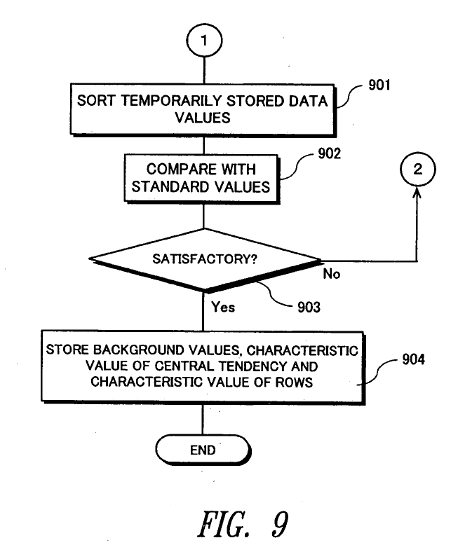

Next, a second embodiment of the invention will be described. In the second embodiment, suitable parameters are calculated for precluding periodicity. FIGS. 8 and 9 are flowcharts schematically illustrating the processing according to the second embodiment. In the second embodiment, too, the data are arranged in order of spotting at the time of preparing a DNA chip by using the spotter like in the initial correction described with reference to FIG. 6. Further, not being limited to the transverse rows, the similar processing can also be executed even for the vertical columns like in the example of FIG. 6.

In this processing, there are obtained data of predetermined rows (step 801), and a characteristic value of central tendency of the row is calculated from the data values of the row (step 802). Here, the median value may be used, or the characteristic value may be calculated from an average of logarithmic values of the remaining data after the upper limit and the lower limit have been removed. Next, a background value of the row is set (step 803). It is considered that the background value that is set is proportional to the characteristic value of central tendency calculated at step 802. Namely, it is considered that the background value is αMi for a characteristic value Mi (i is a row number) of central tendency of a given row.

Next, the data values from which the background value is subtracted are converted into a logarithmic form (steps 804, 805). The data values that are smaller than the background value cannot be converted into logarithmic values. It is desired that such data are indicated to be smaller than a limit of measurability, and are so displayed on the screen of the display unit. Then, from the logarithmic value is subtracted the characteristic value Mi of central tendency or a value obtained by subtracting the background value from the characteristic value of central tendency (step 806). Further, a characteristic value of variation (second characteristic value) is set, and the above subtracted value is divided by this second characteristic value (step 807). As for the characteristic value of variation, it is desired, for example, to draw a graph with the corresponding standard values on the x-axis and the above divided values which have been sorted on the y-axis, and take the values in the range (e.g., range of upper 60% to 90%) closest to y=x to be the characteristic value of variation (second characteristic value) σ.

Namely, through steps 801 to 808, (log (X−αMi)−Mi)/σ of a given column i is calculated. This processing is executed for each of the columns (steps 809, 810). Further, these data are temporarily stored in the data buffer 30.

Thereafter, the temporarily stored data values are sorted and are compared with the corresponding standard values (steps 901, 902). Here, too, a graph is drawn with the corresponding standard values on the x-axis and the sorted data values on the y-axis, and it is judged whether the plotted points are approximate to y=x. When they are sufficiently close (yes at step 903), the background values (αMi) of the respective rows, the characteristic values (Mi) of central tendency and the characteristic values (σ) of variation are stored in the result storage unit 42 (step 904). Whether they are satisfactory or not may be judged from the sum of squares (square errors) of differences between the corresponding standard values and the data values, or from the sum of absolute values of differences.

When the difference exceeds a predetermined range (i.e., when a line connecting the plotted points are deviated from y=x by more than a predetermined amount)(no at step 903), the proportional constant α is changed again, or the characteristic value σ of variation is changed accordingly, and the processes starting with step 801 are repeated. According to this embodiment, the background value of the i-th column of the DNA chip is set to be αMi and the characteristic value of central tendency is set to be Mi, making it possible to eliminate bias in the chip causing marked differences between each of the columns.

Next, a third embodiment of the invention will be described. In the first embodiment, the background value (σ), characteristic value (μ) of central tendency and characteristic value (σ) of variation are calculated based on the data values (measured values) actually obtained from the DNA chip. It is, however, probable that noise affecting the median value cannot be neglected. Namely, due to defect in the wet test, the noise level of the hybridization as a whole is often heightened. As the noise level approaches the median value, it becomes difficult to apply even the robust method of the first embodiment. Here, the noise stands for components that happen to be contained in the individual data and is considered to stem from measuring errors or from errors in the amount of spots. The noise is a concept corresponding to the signal, and the raw data obtained from the DNA chip can be considered to be the sum of noise and signal. Further, the background can be defined to be a portion that is contained in the individual data signals but that does not stem from the RNA in the sample. Accordingly, the signal can be comprehended as the sum of a portion stemming from the RNA and the background.

Even when the noise level is high as described above, the-data of higher signals becomes sufficiently larger than the noise level from the nature of normal logarithmic distribution. If a suitable true characteristic value of central tendency can be found, then the data can be analyzed. If the background can be obtained, the characteristic value of central tendency can be found by a method of try and improve. However, a relationship between the background and the characteristic value of central tendency is not known. In the normal logarithmic distribution using three parameters introduced by the invention, it is difficult to find the two parameters by the above method due to the amount of calculation and the selection from among more than one solution.

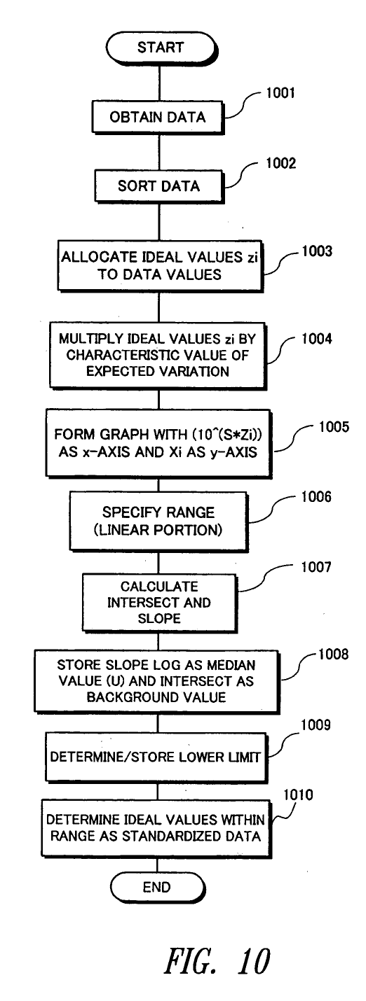

From a combination of a chip and a sample, therefore, if a normal logarithmic distribution can be expected without noise, then, the values can be obtained by the following method. FIG. 10 is a flowchart illustrating the processing according to the third embodiment.

According to the third embodiment as shown in FIG. 10, the original data of the DNA chip are obtained from the data buffer 30 (step 1001) and are sorted and rearranged in order of increasing values or decreasing values (step 1002). The sorted data, too, are stored in the data buffer 30. Then, ideal values Zi (i=1, 2, - - - ) of normal logarithmic distribution are assigned to the sorted data values (step 1003). The ideal values Zi can be calculated by nearly the same method as the one for calculating the standard values of the first embodiment (see step 403). If briefly described here, again, m(i) is, first, calculated as follows:
m(i)=(i−0.3175)/(n+0.365)
where n is the number of data items, and i is a natural number of from 1 to n.

Then, to the M(i) that are calculated are applied an inverse function F^{31 1}(r) of the normal distribution function. The values Zi that are calculated correspond to the data values. The standard values, too, are utilized for the subsequent processing, and are stored in the data buffer 30.

The ideal values Zi thus calculated are, then, multiplied by the characteristic value (s) of variation expected for Zi (step 1004). It is considered that the characteristic value of variation does not fluctuate for each of the experiments, and can be estimated to some extent.

Next, a graph is drawn with 10 raised to a power of the above value obtained by the above multiplication. (i.e., 10^{(s Zi)}) on the x-axis and the measured value xi on the y-axis (step 1005). In this graph, the linear portion can be considered to be a reliable region. Therefore, if the user makes a reference to a graph that is displayed and selects a straight portion (specifies the range)(step 1006), then, a intersect of the graph and an slope are calculated (step 1007). The obtained slope in a logarithmic form is stored as a characteristic value (u) of central tendency and the intersect is stored as a background value (g).

The characteristic value of the thus obtained central tendency and the effectiveness of the background will now be briefly described. In the standardization (Z-standardization) using three parameters of the present invention, Zi can be expressed by the following formula,
Zi={log(xi−g)−u}/s
where Zi is an ideal value, xi is the measured value corresponding thereto, and g, u and s are the background value, the characteristic value of central tendency and the characteristic value of variation, respectively.

If the above formula is solved for xi, then,
xi=(10^{u})(10^{(sZi)})+g

If the values are plotted with (10^{(s Zi)}) as the x-axis and xi as the y-axis, then, there is obtained a line which is linear within a predetermined range. In this straight line, 10^{u} is the slope. Therefore, a logarithm of slope makes it possible to obtain a characteristic value u of central tendency. The background value g obtained as described above, the characteristic value u of central tendency and characteristic value s of variation are stored in the result storage unit 42 (step 1008).

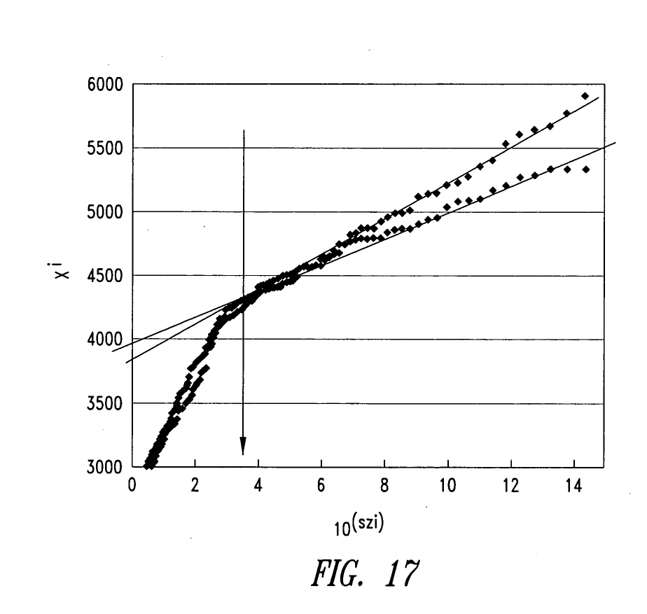

The third embodiment is applied to the data in a state where it is difficult to conduct the analysis by the robust method due to a high noise level. Therefore, the range (lower limit value) of data values that can be utilized is calculated as described below. Here, a range (or a lower limit value) where the linearity is maintained is found in a graph in which the values are plotted with (10^{(s Zi)}) obtained at step 1005 as the x-axis and xi as the y-axis (step 1009). The thus determined lower limit values, too, are stored in the result storage unit 42. FIG. 17 is a graph drawn by plotting the values with (10^{(s Zi)}) as the x-axis and xi as the y-axis of the data stemming from the DNA. In FIG. 17, a graph illustrates a mass of twelve data values hit by twelve pins. Here, (10^{(sZi)}) loses linearity at about 3.5. In this example, s≅0.78 and, hence, it is learned that the lower limit value of Zi is about 0.7.

Next, the data values having ideal values within the range (i.e., not smaller than the lower limit) are taken out. It is desired that those that lie outside the range are indicated to be those smaller than the measurable limit and are so displayed on the screen of the display unit. On the other hand, the ideal values that are taken out are indicated as standardized data values (step 1010).

According to the third embodiment, it is possible to standardize the data based on acquiring a normal logarithmic distribution even when it is not possible to apply the method of the first embodiment because of a high noise level. It is further possible to specify the lower limit of data values that can be utilized.

It goes without saying that the present invention is in no way limited to the above-mentioned embodiments only but can be modified in a variety of ways within a scope described in the claims and that such modifications are also encompassed in the scope of the invention.

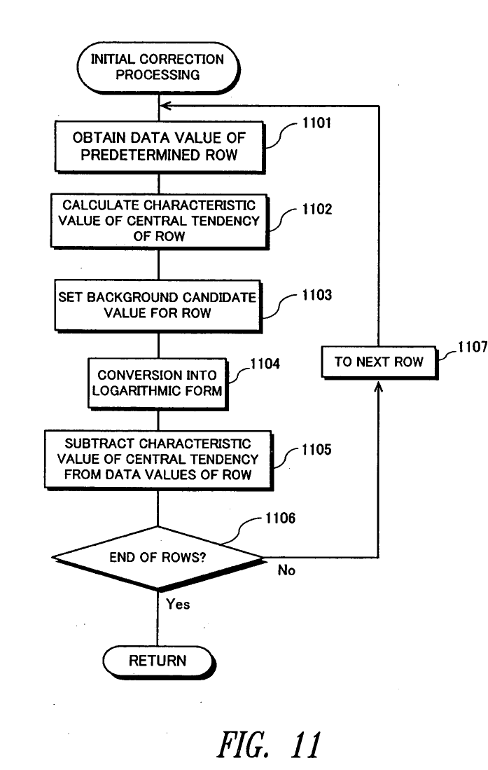

The initial correction processing, for example, is not limited to the one described above. FIG. 11 is a flowchart illustrating another example of the initial correction processing. In the example shown in FIG. 11, too, the processing is conducted for:excluding the variation pattern of data for each of the columns or rows. Here, the background values are determined for each of the columns based on the characteristic value of central tendency (see steps 1101 to 1103), and the subtracted values obtained by subtracting the preset background value from the data values are converted into a logarithmic form (step 1104). Then, the characteristic value of central tendency is subtracted from the logarithmic values (step 1105). Here, too, the median data value for each of the columns may be used as the characteristic value of central tendency, or an average value of the remaining data values after the upper limit and lower limit are removed may be used. It is further desired to make the characteristic value the multiplication product of a proportional constant and the background value. This processing is executed up to the end of the rows (see steps 1106 and 1107) making it possible to eliminate fluctuation in the production of chips.

In the above embodiment, further, the data obtained from the DNA chip are processed to obtain data that can be analyzed by such processes as comparison. Not being limited to the DNA chips only, however, the invention can also be applied even to the so-called protein chips. That is, the invention can also be applied even to the data obtained by labeling coarse proteins in the sample of the protein chip and applying them to an antibody chip.

Further, the invention is not limited to the DNA chips or protein chips, but can similarly be applied even to the data of the amount of gene expression obtained by any method, such as the data obtained by securing genes such as DNA to the microbeads.

As the DNA chip for offering the data to which the data processing method of the invention can be applied, it is desired to use the one in which the spot positions of clone of cDNA are separated away in a random fashion from the origin or expression of the clones. When it is attempted to spot a clone stemming from a single tissue or to spot a limited kind of clone, it is desired to spot a plurality of kinds of clones that are selected in a random fashion as a control for measuring the characteristic value of central tendency of data (or characteristic value of variation).

According to the present invention, it is made possible to provide a data processing method capable of highly precisely analyzing the data obtained from the DNA chips.

Thus, the invention is to be limited by the scope of the claims that follow and the equivalents thereof.

1. A method of processing gene expression data to obtain data that can be analyzed by processing array data obtained based on the amount of expression of genes, comprising the steps of:
obtaining the array data, sorting the data values of the obtained array data, picking a predetermined number of data values from the sorted data values each at a predetermined interval from each other, and temporarily storing them in storage means;
selecting a plurality of background candidates and temporarily storing them in the storage means;
subtracting the values of the background candidates from the data values that are picked up to obtain subtracted values, obtaining logarithmic values by subjecting the subtracted values to logarithmic conversion, and temporarily storing the logarithmic values in the storage means;
calculating normal distribution standard values corresponding to the logarithmic values;
calculating indexes of differences between the logarithmic values and the standard values for the background candidates;
narrowing the range of the background candidate values based on the indexes;
repeatedly obtaining and calculating the indexes of the differences between the subtracted values and logarithmic values, and narrowing the background candidate values, to determine the background value; and
standardizing the temporarily stored logarithmic values by relating them to the determined background value, and storing the standardized values in the storage means.

2. A method of processing gene expression data to obtain data that can be analyzed by processing array data obtained based on the amount of expression of genes, comprising the steps of:
obtaining the array data, sorting the data values of the obtained array data, obtaining a predetermined number of data values from the sorted data values each at a predetermined interval from each other, and temporarily storing them in storage means;
determining a background value ν and storing it in the storage means;
obtaining subtracted values, which are the data values from which the background value is subtracted, into a logarithmic form to obtain logarithmic values, and temporarily storing them in the storage means;
referring to the logarithmic values to calculate a characteristic value μ of central tendency and a characteristic value σ of variation, and storing them in the storage means; and
calculating z=(log (x−ν)−μ)/σ as standard values z for the data values x, and storing the calculated standard values z in the storage means.

3. The method as set forth in claim 2, wherein the step of determining the background value ν includes the steps of:
selecting a plurality of background candidates and temporarily storing them in the storage means;
subtracting the values of the background candidates from the data values that are obtained to obtain subtracted values, obtaining logarithmic values by subjecting the subtracted values to logarithmic conversion, and temporarily storing the logarithmic values in the storage means;
calculating normal distribution standard values corresponding to the logarithmic values;
calculating indexes of differences between the logarithmic values and the standard values for the background candidates;
narrowing the range of the background candidate values based on the indexes; and
repeatedly obtaining the subtracted values and logarithmic values, calculating the indexes of the differences, and narrowing the background candidate values, to determine the background value.

4. The method as set forth in claim 2, wherein the step of calculating a characteristic value μ of central tendency and a characteristic value ν of variation includes the steps of:
calculating standard values corresponding to the logarithmic values;
comparing the logarithmic values with the standard values to find a range in which the ratio of the two shifts nearly at a constant rate;
calculating the slope of the straight line formed in the above range when the standard value is considered to be the x-axis and the logarithmic value to be the y-axis, as well as calculating a y-intersect; and
making the calculated y-intersect the characteristic value μ of central tendency and making the slope the characteristic value σ of variation.

5. The method as set forth in claim 1, further comprising the steps of:
rearranging the order of data values from the order of spots arranged on the chip, and temporarily storing them in this order in the storage means;
calculating the indexes of the variation pattern in data values among each of the columns or rows in which the spots are arranged in the chip;
calculating the median value of data values for each of the columns or rows based on the indexes when each of the columns or rows has a distinctive characteristic; and
dividing the data values by the corresponding median values to obtain divided values, and temporarily storing them in the storage means;
wherein the divided values which are temporarily stored are used for the operation as values corresponding to the data values of the array data.

6. The method as set forth in claim 2, wherein the step of calculating a characteristic value μ of central tendency and a characteristic value σ of variation includes the steps of:
calculating standard values corresponding to the logarithmic values;
comparing the logarithmic values with the standard values to find a range in which the ratio of the two shifts nearly at a constant rate;
calculating the slope of a straight line formed in the above range when the standard value is considered to be the x-axis and the logarithmic value to be the y-axis, as well as calculating a y-intersect; and
making the calculated y-intersect the characteristic value μ of central tendency and making the slope the characteristic value a of variation.

7. The method as set forth in claim 1, further comprising the steps of:
rearranging the order of data values from the order of spots arranged on the chip, and temporarily storing them in this order in the storage means;
calculating the indexes of the variation pattern in data values for each of the columns or the rows in which the spots are arranged in the chip;
calculating the median value of data values for each of the columns or rows based on the indexes when there is a distinctive characteristic for each of the columns or rows; and
dividing the data values by the corresponding median values to obtain divided values, and temporarily storing them in the storage means;
wherein the divided values which are temporarily stored are used for the operation as values corresponding to the data values of the array data.

8. The method as set forth in claim 2, further comprising the steps of:
rearranging the order of data values from the order of spots arranged on the chip, and temporarily storing them in this order in the storage means;
calculating indexes of the variation pattern in data values for each of the columns or rows in which the spots are arranged in the chip;
calculating the median value of data values for each of the columns or rows based on the indexes when there is a distinctive characteristic for each of the columns or rows; and
dividing the data values by the corresponding median values to obtain divided values, and temporarily storing them in the storage means;
wherein the divided values which are temporarily stored are used for the operation as values corresponding to the data values of the array data.

9. The method as set forth in claim 7, wherein the step of calculating an index that represents the tendency includes a step of calculating an average variation of a particular column or row.

10. The method as set forth in claim 8, wherein the step of calculating an index that represents the variation pattern includes a step of calculating an average variation of a particular column or row.

11. The method as set forth in claim 1, further comprising the steps of:
rearranging the order of data values from the order of spots arranged on the chip, and temporarily storing them in this order in the storage means;
finding a periodicity of data values in the above order; and
calculating subtracted values by subtracting the characteristic value of central tendency of the period from the data values, and temporarily storing them in the storage means;
wherein the temporarily stored subtracted values are used for the operation as values corresponding to the data values of the array data.

12. The method as set forth in claim 2, further comprising the steps of:
rearranging the order of data values from the order of spots arranged on the chip, and temporarily storing them in this order in the storage means;
finding a periodicity of data values in the above order; and
calculating subtracted values by subtracting the characteristic value of central tendency of the period from the data values, and temporarily storing them in the storage means;
wherein the temporarily stored subtracted values are used for the operation as values corresponding to the data values of the array data.

13. The method as set forth in claim 1, further comprising the steps of:
rearranging the order of data values from the order of spots arranged on the chip;
calculating characteristic values of central tendency of data values of the columns or rows on where the spots are arranged in the chip for each of the columns or rows;
setting background values corresponding to the spots belonging to the columns or rows based on the characteristic value of central tendency, and calculating subtracted values by subtracting the background values from the data values of the spots;
converting the subtracted values into a logarithmic form to obtain logarithmic values; and
subtracting characteristic values of central tendency of said logarithmic values of the columns or rows and temporarily storing the subtracted values in the storage means;
wherein the temporarily stored subtracted values are used for the operation as values corresponding to the data values of the array data.

14. The method as set forth in claim 2, further comprising the steps of:
rearranging the order of data values from the order of spots arranged on the chip;
calculating characteristic values of central tendency of data values of the columns or rows on where the spots are arranged in the chip for each of the columns or rows;
setting background values of the spots belonging to the columns or rows based on the characteristic value of central tendency, and calculating subtracted values by subtracting the background values from the data values of the spots;
converting the subtracted values into a logarithmic form to obtain logarithmic values; and
subtracting characteristic values of central tendency of said logarithmic values of the columns or rows and temporarily storing the subtracted values in the storage means;
wherein the temporarily stored subtracted values are used for the operation as values corresponding to the data values of the array data.

15. A method of processing gene expression data to obtain data that can be analyzed by processing array data obtained based on the amount of expression of genes, comprising the steps of:
calculating the characteristic value of central tendency of data values of the columns or rows where the spots are arranged in the chip for each of the columns or rows;
setting a candidate for the background value of the spot belonging to the column or row based on the characteristic value of central tendency, and calculating a subtracted value by subtracting the background candidate value from the data values of the spot;
converting the subtracted values into a logarithmic form to obtain logarithmic values;
calculating a characteristic value of central tendency of the logarithmic value of the column or the row, and subtracting the characteristic value from the logarithmic values to calculate the second subtracted values;
dividing the data values by the characteristic value of variation calculated based on the second subtracted value of the column or the row to obtain divided values, and temporarily storing them in the storage means;
comparing the divided values with the corresponding standard values, and making the background candidate value which minimizes the index of difference between them the background value ν; and
storing the background value ν, a characteristic value μ of central tendency of the background value ν and a characteristic value σ of variation in the storage means.

16. A method of processing gene expression data to obtain data that can be analyzed by processing array data obtained based on the amount of expression of genes, comprising the steps of:
obtaining the array data, sorting the data values of the obtained array data, and temporarily storing the sorted data in the storage means;
calculating normal distribution standard values corresponding to the sorted data values;
setting a characteristic value s of variation of the data value, storing it in the storage means, and multiplying the standard values by the characteristic value s of variation to obtain multiplied values;
comparing the data values with the multiplied values to find a range in which the ratio of the two shifts at a constant rate;
calculating the slope of a straight line formed in the above range, where the multiplied value is considered to be the x-axis and the logarithmic value to be the y-axis and calculating a y-intersect; and
making the natural logarithm of the slope the characteristic value μ of central tendency and the intersect as a background value g, and storing them in the storage means.

17. The method as set forth in claim 16, further comprising the steps of:
solving xi in compliance with,
xi=(10^{u}) (10^{(s Zi)})+g
where Zi is an i-th standard value,
and temporarily storing it in the storage means; and
finding a lower limit value where xi can be used, and storing it in the storage means.

18. A program that can be read by a computer to operate the computer to obtain data that can be analyzed by processing the array data obtained based on the amount of expression of genes, the program working to have the computer execute the steps of:
obtaining the array data, sorting the data values of the obtained array data, picking up a predetermined number of data values from the sorted data values each at a predetermined interval from each other, and temporarily storing them in storage means;
selecting a plurality of background candidates and temporarily storing them in the storage means;
subtracting the values of the background candidates from the data values that are picked up to obtain subtracted values, obtaining logarithmic values by subjecting the subtracted values to logarithmic conversion, and temporarily storing the logarithmic values in the storage means;
calculating normal distribution standard values corresponding to the logarithmic values;
calculating indexes of differences between each of the logarithmic values and the standard values for each of the background candidates;
narrowing the range of the background candidate values based on the indexes;
repeatedly obtaining the subtracted values and logarithmic values, calculating the indexes of the differences, and narrowing the background candidate values, to determine the background value; and
standardizing the logarithmic values temporarily stored by relating them to the determined background value, and storing the standardized values in the storage means.

19. A program that can be read by a computer to operate the computer to obtain data that can be analyzed by processing the array data obtained based on the amount of expression of genes, the program working to have the computer execute the steps of:
obtaining the array data, sorting the data values of the obtained array data, picking up a predetermined number of data values from the sorted data values each at a predetermined interval from each other, and temporarily storing them in storage means;
determining a background value ν and storing it in the storage means;
converting subtracted values which are the data values from which the background value is subtracted into a logarithmic form to obtain logarithmic values, and temporarily storing them in the storage means;
referring to the logarithmic values to calculate a characteristic value μ of central tendency and a characteristic value σ of variation, and storing them in the storage means; and
calculating z=(log (x−ν)−μ)/σ as standard values z for the data values x, and storing the calculated standard values z in the storage means.

20. The program as set forth in claim 19, wherein the computer in the step for determining the background value ν executes the steps of:
selecting a plurality of background candidates and temporarily storing them in the storage means;
subtracting the values of the background candidates from the data values that are picked up to obtain subtracted values, obtaining logarithmic values by subjecting the subtracted values to logarithmic conversion, and temporarily storing the logarithmic values in the storage means;
calculating normal distribution standard values corresponding to the logarithmic values;
calculating indexes of differences between the logarithmic values and the standard values for the background candidates;
narrowing the range of the background candidate values based on the indexes; and
repeatedly obtaining the subtracted values and logarithmic values, calculating the indexes of the differences, and narrowing the background candidate values to determine a background value.

21. The program as set forth in claim 19, wherein the computer in the step of calculating a characteristic value μ of central tendency and a characteristic value σ of variation executes the steps of:
calculating standard values corresponding to the logarithmic values;
comparing the logarithmic values with the standard values to find a range in which the ratio of the two shifts nearly at a constant rate;
calculating the slope of a straight line formed in the above range, where the standard value is considered to be the x-axis and the logarithmic value to be the y-axis, as well as calculating a y-intersect; and
making the calculated y-intersect the characteristic value μ of central tendency and making the slope the characteristic value σ of variation.

22. The program as set forth in claim 16, wherein the computer executes the steps of:
rearranging the order of data values from the order of spots arranged on the chip, and temporarily storing them in this order in the storage means;
calculating the indexes of the variation pattern in data values for each of the columns or rows in which the spots are arranged in the chip;
calculating the median value of data values for each of the columns or rows based on the indexes when there is a distinctive characteristic for each of the columns or rows; and
dividing the data values by the corresponding median values to obtain divided values, and temporarily storing them in the storage means;
wherein the divided values which are temporarily stored are used for the operation as values corresponding to the data values of the array data.

23. The program as set forth in claim 19, wherein the computer executes the steps of:
rearranging the order of data values from the order of spots arranged on the chip, and temporarily storing them in this order in the storage means;
calculating the indexes of the variation pattern in data values for each of the columns or rows in which the spots are arranged in the chip;
calculating the median value of data values for each of the columns or rows based on the indexes when there is a distinctive characteristic for each of the columns or rows; and
dividing the data values by the corresponding median values to obtain divided values, and temporarily storing them in the storage means;
wherein the divided values which are temporarily stored are used for the operation as values corresponding to the data values of the array data.

24. The program as set forth in claim 22, wherein the computer in the step of calculating an index that represents the variation pattern executes a step of calculating the average variation of a particular column or row.

25. The program as set forth in claim 23, wherein the computer in the step of calculating an index that represents the variation pattern executes a step of calculating an average variation of a particular column or row.

26. The program as set forth in claim 18, wherein the computer executes the steps of:
rearranging the order of data values from the order of spots arranged on the chip, and temporarily storing them in this order in the storage means;
finding a periodicity of data values in the above order; and
calculating subtracted values by subtracting the characteristic value of central tendency of the period from the data values, and temporarily storing them in the storage means;
wherein the temporarily stored subtracted values are used for the operation as values corresponding to the data values of the array data.

27. The program as set forth in claim 19, wherein the computer executes the steps of:
rearranging the order of data values from the order of spots arranged on the chip, and temporarily storing them in this order in the storage means;
finding a periodicity of data values in the above order; and
calculating subtracted values by subtracting the characteristic value of central tendency of the period from the data values, and temporarily storing them in the storage means;
wherein the temporarily stored subtracted values are used for the operation as values corresponding to the data values of the array data.

28. The program as set forth in claim 18, wherein the computer executes the steps of:
rearranging the order of data values from the order of spots arranged on the chip;
calculating characteristic values of central tendency of data values of the columns or the rows on where the spots are arranged in the chip for each of the columns or rows;
setting background values corresponding to the spots belonging to the columns or rows based on the characteristic value of central tendency, and calculating subtracted values by subtracting the background values from the data values of the spots;
converting the subtracted values into a logarithmic form to obtain logarithmic values; and
subtracting characteristic values of central tendency of said logarithmic values of the columns or rows and temporarily storing the subtracted values in the storage means;
wherein the temporarily stored subtracted values are used for the operation as values corresponding to the data values of the array data.

29. The program as set forth in claim 19, wherein the computer executes the steps of:
rearranging the order of data values from the order of spots arranged on the chip;
calculating characteristic values of central tendency of data values of the columns or the rows on where the spots are arranged in the chip for each of the columns or rows;
setting background values corresponding to the spots belonging to the columns or rows based on the characteristic value of central tendency, and calculating subtracted values by subtracting the background values from the data values of the spots;
converting the subtracted values into a logarithmic form to obtain logarithmic values; and
subtracting characteristic values of central tendency of said logarithmic values of the columns or rows and temporarily storing the subtracted values in the storage means;
wherein the temporarily stored subtracted values are used for the operation as values corresponding to the data values of the array data.

30. A program that can be read by a computer to operate the computer to obtain data that can be analyzed by processing the array data obtained based on the amount of expression of genes, the program working to have the computer execute the steps of:
calculating characteristic values of central tendency of data values of the columns or the rows on where spots are arranged in the chip for each of the columns or rows;
setting candidates for background values of the spots belonging to the column or the row based on the characteristic values of central tendency, and calculating subtracted values by subtracting the background candidate values from the data values of the spots;
converting the subtracted values into a logarithmic form to obtain logarithmic values;
calculating characteristic values of central tendency of the logarithmic values of the columns or the rows and subtracting the characteristic values from the logarithmic values to calculate second subtracted values;
obtaining divided values by dividing the data values by the characteristic value of variation calculated based on the second subtracted values of the column or the row, and temporarily storing them in the storage means;
comparing the divided values with the corresponding standard values and making the background candidate value which minimizes the index of difference between them the background value ν; and
storing the background value ν, the characteristic value μ of central tendency of the background value ν and the characteristic value σ of variation in the storage means.

31. A program that can be read by a computer to operate the computer to obtain data that can be analyzed by processing the array data obtained based on the amount of expression of genes, the program working to have the computer execute the steps of:
obtaining the array data, sorting the data values of the obtained array data, and temporarily storing the sorted data in the storage means;
calculating normal distribution standard values corresponding to the sorted data values;
setting a characteristic value s of variation of the data values, storing it in the storage means, and multiplying the standard values by the characteristic value s of variation to obtain multiplied values;
comparing the data values with the multiplied values to find a range in which the ratio of the two shifts at a constant rate;
calculating the slope of the straight line formed in the above range, where the multiplied value is considered to be the x-axis and the logarithmic value to be the y-axis and calculating a y-intersect; and
making the natural logarithm of the slope the characteristic value μ of central tendency and making the intersect the background value g, and storing them in the storage means.

32. The program as set forth in claim 31, wherein the computer executes the steps of:
solving xi in compliance with,
xi=(10^{u})(10^{(sZi)})+g
where Zi is an i-th standard value,
and temporarily storing it in the storage means; and
finding a lower limit value where xi can be used and storing it in the storage means.
